(19) United States
(12) Patent Application Publication
(10) Pub. No.: US 2002/0150975 A1
(43) Pub. Date: Oct. 17, 2002

(54) Snake toxin and its use as pharmaceutical

(76) Inventors: Christoph Methfessel, Wuppertal (DE); Viktor Tsetlin, Moskau (RU); Yuri Utkin, Moskau (RU)

Correspondence Address: CONNOLLY BOVE LODGE & HUTZ, LLP, 1220 N MARKET STREET, P O BOX 2207, WILMINGTON, DE, 19899

(21) Appl. No.: 09/911,276

(22) Filed: Jul. 23, 2001

(30) Foreign Application Priority Data
Jul. 24, 2000 (DE) 10035854.3

(51) Int. Cl. C07K 14/81; C12P 21/02; C12N 5/06

(52) U.S. Cl. 435/69.1; 530/324; 435/325; 435/320.1

(57) ABSTRACT

The present invention relates to a peptide having the amino acid sequence

```
Leu Thr Cys Leu Asn Cys Pro Glu Met Phe
    1  5  10
Cys Gly Lys Phe Gln Ile Cys Arg Asn Gly
                              15  20
Glu Lys Ile Cys Phe Lys Lys Leu His Gln
                              25  30
Arg Arg Pro Leu Ser Trp Arg Tyr Ile Arg
                              35  40
Gly Cys Ala Asp Thr Cys Pro Val Gly Lys
                              45 50
Pro Tyr Glu Met Ile Glu Cys Cys Ser Thr
                                55  60
Asp Lys Cys Asn Arg
                                65
```

or alleles derived therefrom, or peptides containing essential parts of the peptide (I), to a process for the preparation thereof and the use thereof for producing a pharmaceutical for the treatment of diseases of which therapy is possible through inhibition of the α7-nACh receptor, in particular the treatment of cancer such as small cell lung carcinoma.

The present invention relates to a novel snake toxin and to peptides or compounds derived therefrom, to the isolation thereof and to the use thereof for inhibiting the growth of cancer cells, specifically of small cell lung carcinoma (SCLC).

Nicotinergic acetylcholine receptors (nAChR) are an important class of ligand-controlled ion channels. They are exceptionally widespread in the human body and in the animal kingdom and are involved in many important processes of signal transmission and cell recognition in the organism (cf. Lindstrom, Jon M., Nicotinic acetylcholine receptors, in: Ligand-Voltage-Gated Ion Channels, 153-75, Editor(s): North, R. Alan, CRC Press (1995); Bertrand D. and Changeux J.-P., Nicotinic Receptor: an allosteric protein specialized for intercellular communication, Seminars in The Neurosciences 7, 75-90 (1995)).

The nAChR known to date in the human body can be attributed to about 15 unambiguously identified and molecular biologically characterized DNA sequences or genes. Each of these genes codes for a protein which can be identified by a number of characteristic chemical, biochemical and structural features as a potential subunit of an nAChR complex (Lindstrom J. M., Purification and Cloning of Nicotinic Acetylcholine Receptors, p. 3-23, in: Arneric S. P. and Brioni J. D., eds., Neuronal Nicotinic Receptors: Pharmacology and Therapeutic Opportunities, Wiley-Liss (1998)).

A functional nAChR complex in the cell membrane of a human body cell consists of five such nAChR proteins. These five subunits of such a receptor complex may be encoded by different nAChR genes and, as a rule, the type and function of a cell determines which combinations of nAChR subunits contribute to the expression of functional nAChR pentamers in its membrane (Ramirez-Latorre J., Crabtree G., Turner J., Role L., Molecular Composition and Biophysical Properties of Nicotinic Receptors, p. 43-64, in: Arneric S. P. and Brioni J. D., eds., Neuronal Nicotinic Receptors: Pharmacology and Therapeutic Opportunities, Wiley-Liss (1998)).

It is known that the α1, β1, γ, δ, and ε subunits preferentially occur in muscle cells and are responsible there for transmission of the nerve impulse to the muscle. This is the first subfamily of nAChR in the human body. The α2, α3, α4, α5 and α6 subunits, and the β2, β3 and β4 subunits are preferentially expressed in nerve cells and neuroendocrine cells, where they form, in various combinations, functional nAChR complexes and have important functions, the details of which have not yet been fully elucidated, in cellular signal transmission. These “neuronal” nAChR form the second family of nicotinergic receptors in the body. Finally, there are the α7 and α9 subunits which, in contrast to all other nAChR subunits, can form functional nAChR channels even on their own, that is to say as homopentameric protein complexes, although it is not ruled out that α7 or α9 subunits may also form functional nAChR together with other subunits from the first or second family (Peng X., Katz M., Gerzanich V., Anand R., Lindstrom J., Human α7 acetylcholine receptor: cloning of the α7 subunit from the SH-SY5Y cell lines and determination of pharmacological properties of native receptors and functional α7 hormones expressed in Xenopus oocytes, Molecular Pharmacology 45, 546-554 (1994); Alkondon M., Albuquerque E. X., Diversity of Nicotinic Acetylcholine Receptors in Rat Hippocampal Neurons, I. Pharmacological and functional evidence for distinct structural subtypes., J. Pharmacol. Expt. Ther. 265, 1455-1473 (1993); Bertrand D., Bertrand S., Ballivet M., Pharmacological properties of the homomeric alpha-7 receptor, Neurosci. Lett. 146, 87-90 (1992); Castro N. G., Albuquerque E. X., Alpha-bungarotoxin sensitive hippocampal nicotinic receptor channel has a high calcium permeability, Biophysical Journal 68, 516-524 (1995), Zhang Z. W., Vijayaraghavan S., Beg D. K., Neuronal acetylcholine receptors that bind alpha-Bungarotoxin with high affinity function as ligand-gated ion channels, Neuron 12, 167-177 (1994)).

Functional nAChR complexes containing at least one α7 subunit are referred to herein as “α7-nAChR”. They are very widespread in the central nervous system (CNS) but also occur in other tissues and cells, for example also in epithelial, skin or secretory cells and particularly in neuroendocrine cells, including the lung, inter alia.

Compared with the other nAChR types, α7-nAChR are distinguished by a number of special properties. They can be activated not only by the natural messenger acetylcholine but also by its natural degradation product choline (Albuquerque E. X., Pereira E. F. R., Braga M. F. M., Alkondon M., Contribution of nicotinic receptors to the function of synapses in the central nervous system: The action of choline as a selective agonist of, 7 receptors, J. Physiology (Paris) 92, 309-316 (1998)). When they are activated they allow not only, and primarily, sodium to flow through the cell membrane but also doubly charged calcium, and it is known that calcium entering in this way is able to initiate a whole series of biochemical, regulatory, and growth-promoting effects in cells. It is also possible for α7-nAChR to be blocked or inactivated by certain snake toxins, gastropod toxins or plant poisons which have only slight effect, or none, on other neuronal nAChR (Bertrand D., Bertrand S., Ballivet M., Pharmacological properties of the homomeric alpha-7 receptor, Neurosci. Lett. 146, 87-90 (1992); Castro N. G., Albuquerque E. X., Alpha-bungarotoxin sensitive hippocampal nicotinic receptor channel has a high calcium permeability, Biophysical Journal 68, 516-524 (1995)).

It is known that the venom sera from various snakes contain peptide toxins which can biochemically recognize and bind nAChR and functionally block and inactivate it (called alpha-toxins). The snake toxins known to be able to block α7-nAChR include, for example, alpha-bungarotoxin (αBgTx) from Bungarus multicinctus or alpha-cobratoxin (αCbTx) from Naja kaouthia (formerly Naja siamensis) (Hucho F., Peptide Toxins acting on the Nicotinic Acetylcholine Receptor. Chap. 16, p. 577-610 in: Handbook of Experimental Pharmacology (1992), Loring, R. H., The molecular basis of curaremimetic snake neurotoxin specificity for neuronal nicotinic receptor subtypes, J. Toxicology—Toxin Reviews 12 (2), p. 105-153 (1993)). An important feature of the protein structure of such alpha-toxins is the formation of three loops or fingers, and these toxins are also accordingly referred to as three-finger toxins (Menez, A., Functional architectures of animal toxins: a clue to drug design?, Toxicon 36 (11), 1557-1572 (1998); Tsetlin V., Snake venom alpha-neurotoxins and other ‘three-finger’ proteins, Eur. J. Biochem. 264 (2), 281-286 (1999)). These toxins are further differentiated into short-sequence and long-sequence toxins according to the features of their sequences and the number of disulphide linkages (Tsetlin V., Snake venom alpha-neurotoxins and other ‘three-finger’ proteins, Eur. J. Biochem. 264 (2), 281-286 (1999)).

It must be emphasized that the abovementioned toxins act on and block not only α7-nAChR but also, mainly, the nAChR in the muscles, so that these toxins lead to a potent and long-lasting paralysis of muscle and are therefore completely unsuitable for therapeutic use.

It is known that the snake toxins contain not only these nAChR-active alpha-toxins but also a large number of other more or less toxic components which may cause widely differing effects in the body. For many of these components the biological target and the effect on the animal body are only incompletely known, if at all.

One such component, which was described in the literature as long ago as the 1970s but about which otherwise only little was known to date and which, in particular, has not yet been isolated in pure form, is referred to as weak toxin and comprises peptide fractions from the venom of Naja melanoleuca. They showed no noteworthy toxic effect in animal experiments on mice or rats. It has therefore been entirely unclear what biological functions such a weak toxin could have (Carlsson F. H. H., Snake venom toxins, The primary structure of protein S4C 11, A neurotoxin homologue from the venom of forest cobra (Naja melanoleuca), Biochem. Biophys. Acta 400, 310-321 (1975); Joubert F. J., Taljaard N., Snake venoms, The amino acid sequences of two Melanoleuca-type toxins, Hoppe-Seyler's Z. Physiol. Chem. 361, 425-436 (1980), Shafqat J., Siddiqi A. R., Zaidi Z. H., Joernvall H., Extensive multiplicity of the miscellaneous type of neurotoxins from the venom of the cobra Naja naja naja and structural characterization of major components, FEBS Lett. 284, 70-72 (1991)).

Small-cell lung carcinoma SCLC is one of the most malignant cancers and is responsible for about 25% of deaths from lung cancer. It is regarded as incurable. Novel methods or active substances which might inhibit the growth of these cells are therefore of great importance.

Numerous strains of cultivated of SCLC cell lines isolated from SCLC tumours exist. It is possible to use these cells to investigate the biochemical processes influencing the growth and proliferation of these cells, and possibly also to test active substances which might suppress cell growth and thus be suitable as therapeutics for treating SCLC tumours.

It is known that the proliferation of SCLC cells is promoted by activation of nAChR (Maneckjee R., Minna J. D., Opioid and nicotine receptors affect growth regulation of human lung cancer cell lines, Proc. Natl. Acad. Sci. USA, 87, 3294-3298 (1990); Cattaneo M. G., Codignola A., Vincentini L. M., Clementi F., Sher E., Nicotine stimulates a serotonergic autocrine loop in human small cell lung carcinoma, Cancer Res. 53, 5566-5568 (1993); Codignola A., Tarroni P., Cattaneo M. G., Vincentini L. M., Clementi F., Sher E., Serotonin release and cell proliferation are under the control of alpha-bungarotoxin sensitive nicotinic receptors in small-cell lung carcinoma cell lines, FEBS Letters 342, 286-290 (1994)) and that certain substances in tobacco smoke, such as nicotine or the nitrosamine NNK, promote growth and proliferation of SCLC cells in this way (Schuller, H., Nitrosamine-induced Lung Carcinogenesis and Ca2+/Calmodulin Antagonists, Cancer Res. Suppl. 52, 2723s-2726s (1992); Schuller H. M., Orloff M.; Tobacco-specific carcinogenic nitrosamines; Biochem. Pharmacol. 55, 1377-1384 (1998)). It is also known that α7-nAChR occur in malignant small-cell lung cancer cells (SCLC) (Tarroni P., Rubboli F., Chini B., Zwart R., Oortgiesen M., Sher E., Clementi F., Neuronal-type nicotinic receptors in human neuroblastoma and small-cell lung carcinoma cell lines, FEBS Letters 312, 66-70 (1992); Sciamanna M. A., Griesmann G. E., Williams C. L., Lennon V. A., Nicotinic Acetylcholine Receptors of Muscle and Neuronal alpha-7 Types Coexpressed in a Small Cell Lung Carcinoma, J. Neurochemistry 69, 3202-3211 (1997)), so that blockers of this specific subtype of nAChR ought to be of particular interest for the diagnosis or therapy of SCLC disorders.

As has been explained above, however, α7-nAChR can also be activated by endogenously available substances such as acetylcholine and choline, so that the proliferation-promoting effect of the activation of α7-nAChR in the SCLC cells is not necessarily dependent on intake of exogenous α7-nAChR agonists such as nicotine or NNK.

It has already been shown that blockade of α7-nAChR with a suitable active substance can inhibit or suppress proliferation of SCLC cells, at least where this proliferation is promoted by activation of α7-nAChR. It has been described in particular that the proliferation of SCLC cells in vitro is inhibited by snake toxins such as, for example, α-bungarotoxin, α-cobratoxin or conotoxin-ImI (Codignola A., Tarroni P., Cattaneo M. G., Vicentini L. M., Clementi F., Sher E., Serotonin release and cell proliferation are under the control of alpha-bugarotoxin sensitive nicotinic receptors in small-cell lung carcinoma cell lines, FEBS Letters 342, 286-290 (1994); Codignola A., McIntosh J. M., Cattaneo M. G., Vicentini L. M., Clementi F., Sher E., Alpha-conotoxin Imperialis I inhibits nicotine-evoked hormone release and cell proliferation in human neuroendocrine carcinoma cells, Neurosci. Lett. 206, 53-56 (1996)).

It has additionally been described that certain active substances such as morphine are able to induce in SCLC cells the process of apoptosis which leads to death of the cells. This effect can be suppressed with agonists of the nicotine receptor, and this suppression of apoptosis can be reversed with antagonists of the nicotine receptor, such as, for example, the snake toxins already mentioned.

This makes it appear considerable that blockers of nicotine receptors are suitable for controlling the growth and multiplication of SCLC cells or even—where appropriate in combination with other active substances—for leading to death of these cells. Unfortunately, the snake toxins able to exert an inhibitory effect on α7-nAChR and described to date are unsuitable for such a therapy, in particular because these toxins block even more potently the muscle nAChR and thus lead to severe, even life-threatening, paralyses.

A gastropod toxin, conotoxin Im-I, is also known, as mentioned above, and is a potent and selective α7-nAChR blocker. However, this toxin is distinguished by the fact that its binding to α7-nAChR is rapidly reversible so that a continuous presence of the active substance would be necessary for permanent α7-nAChR blockade.

It was therefore the object of the present invention to provide compounds which bind virtually irreversibly to α7-nAChR but do not have the considerable side effects such as, for example, the inhibition of nAChR in muscle to cause severe paralyses, and thus are suitable for the treatment of cancer, in particular of small cell lung cancer (SCLC).

The above object is achieved according to the invention by a peptide having the amino acid sequence

```
Leu Thr Cys Leu Asn Cys Pro Glu Met Phe Cys Gly Lys Phe Gln Ile
    1  5  10  15
Cys Arg Asn Gly Glu Lys Ile Cys Phe Lys Lys Leu His Gln Arg Arg
                          20  25  30
Pro Leu Ser Trp Arg Tyr Ile Arg Gly Cys Ala Asp Thr Cys Pro Val
                  35  40  45
Gly Lys Pro Tyr Glu Met Ile Glu Cys Cys Ser Thr Asp Lys Cys Asn
          50  55  60
Arg  (I)
  65
```

or alleles derived therefrom, for example by compounds containing essential parts of the peptide (I) and having the above effect. This peptide (I) is a constituent of the weak toxin from the venom of Naja kaouthia. It is a novel chemical compound because, in contrast to all similar peptides previously described, it contains a tryptophan residue W36 which does not occur in other peptides of a similar type.

It has been found that the peptides according to the invention are potent and virtually irreversible α7-nAChR blockers. For example, on application of the peptide (I) in the relatively low concentration of 10 μM to rat α7-nAChR which have been expressed in a heterologous expression system (of xenopus oocytes, see literature reference), there is almost complete blockade of the ions influx normally induced in such cells by administration of 100 μM acetylcholine. It is possible to show in a similar type of experiment that this effect also occurs on human α7-nAChR.

It has further been possible to show that the blocking effect of the peptide (I) on α7-nicotine receptor channels is very long-lasting. Whereas blockade of the receptor by other toxins declines again after the toxin is washed out, blockade by the peptide (I) is virtually irreversible and is maintained for a lengthy period after the toxin is washed out.

Thus, the peptides according to the invention are the first peptides which combine a high selectivity for α7-nAChR with a virtually irreversible effect and thus meet the conditions for achieving targeted inhibition of the proliferation of SCLC cells. At the same time, the peptides according to the invention are well tolerated and do not induce any major side effects. This means that the peptides according to the invention are potent but physiologically well tolerated inhibitors of the function of α7-nAChR.

The peptides according to the invention can be employed directly for inhibiting the growth of SCLC cells or tumours in patients.

It is moreover possible for the peptides according to the invention also to be coupled to a marker which then, after binding to the surface of the SCLC cells, is recognized in a second step of the therapy by a cytotoxic active substance such as, for example, a complement system, leading to targeted destruction of these marked cells. It is possible to use as cytotoxic active substance in this case any cytotoxic substance suitable for cancer therapy in humans.

It is also possible to use the peptides according to the invention for assisting the apoptosis-inducing effect of morphine or other opiates and, in this way, to achieve SCLC therapy. As described above, this effect of morphine or other opiates is made possible and/or enhanced by inhibitors of the nicotine receptor such as, for example, the peptides according to the invention.

Since the peptides according to the invention recognize and bind the α7-nAChR on the surface of SCLC cells, they are also suitable for marking the cells and making them identifiable for diagnostic purposes. For this purpose, the peptides can be derivatized by suitable radioactive, fluorescent or other additional chemical groups conventionally used for this purpose, so that cells which carry the α7-nAChR and are probably cancer cells can be differentiated from other harmless cells, or that cancer cells could be assigned unambiguously to the disease of SCLC or another type of cancer.

This also includes chemical modification of the peptides according to the invention so that they can be identified more easily. Suitable chemical derivatizations which could be employed for this purpose are, inter alia: radioactive or fluorescent labellings, ferritin, inorganic nanoparticles, magnetic or other beads, linkers to polymeric substrates, chemical groups carrying recognition features for antibodies, biotin, enzymes, DNA sequences or RNA sequences.

The present invention also includes short-chain peptides which are derived from peptide (I) by up to five individual amino acids being omitted, exchanged for other amino acids or replaced by short sequences of up to five other amino acids of any type and which show an interaction with α7-nAChR corresponding to the peptide (I).

The present invention also includes peptides or proteins which have a longer amino acid sequence, which contain as part of their sequence essential parts of the sequence of the peptide (I), and which are thus able to recognize and bind to α7-nAChR.

The peptides according to the invention may influence cell division or the growth, morphology or physiological behaviour of human cells. It is possible in particular to influence the cell division or the growth, the morphology or the behaviour of those human cells which express nicotinergic acetylcholine receptors in their cell membrane. Among the cells with nicotinergic sensitivity, the invention very particularly relates to the influencing of cells which express the α7-subtype of the nicotinergic receptor. These include, in particular, nerve cells, neuroendocrine cells (such as, for example, chromaffin cells), endocrine cells of the lung, cells of the skin and of epithelial tissue, and very particularly cancer cells too.

The invention quite expressly relates to the influencing of endocrine cells of the lung and of cancer cells which lead to malignant disorders of lung tissue and the airways, and in this connection very specifically small cell lung tumour cells (SCLC).

The invention further relates also to pharmaceutical dosage forms which contain the peptides according to the invention, either alone or together with pharmaceutically suitable excipients.

Dosage forms according to the invention are in particular those having the purpose of taking the peptides according to the invention specifically to that site in the body where the cytostatic or proliferation-inhibiting effect is desired, and is substantially kept away from other regions in the body.

The peptides according to the invention are administered in particular according to the invention in a pharmaceutical form such that the preparation can be inhaled by the patient in such a way that the compounds according to the invention preferentially reach the airways and the lung. In addition, administration according to the invention is particularly in a form such that the average particle size of the pharmaceutical preparation is in the range 100 nm -10 μm, so that on inhalation of an aerosol there is expected to be particularly deep and persistent administration of the compounds in the finer branches of the lung. Other conventional pharmaceutical dosage forms such as, for example, intravenous administration are likewise encompassed by the present invention.

The present invention is further illustrated by Figures. These show:

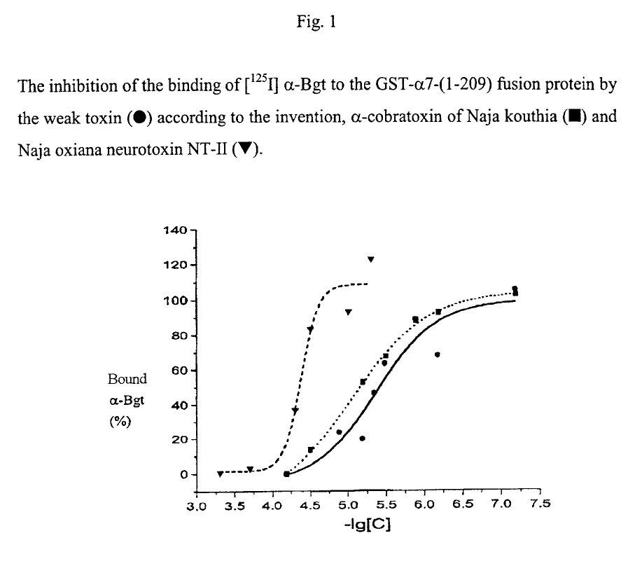

FIG. 1: The inhibition of the binding of [¹²⁵I] α-Bgt to the GST-α7-(1-209) fusion protein by the weak toxin according to the invention, α-cobratoxin of Naja kouthia and Naja oxiana neurotoxin NT-II.

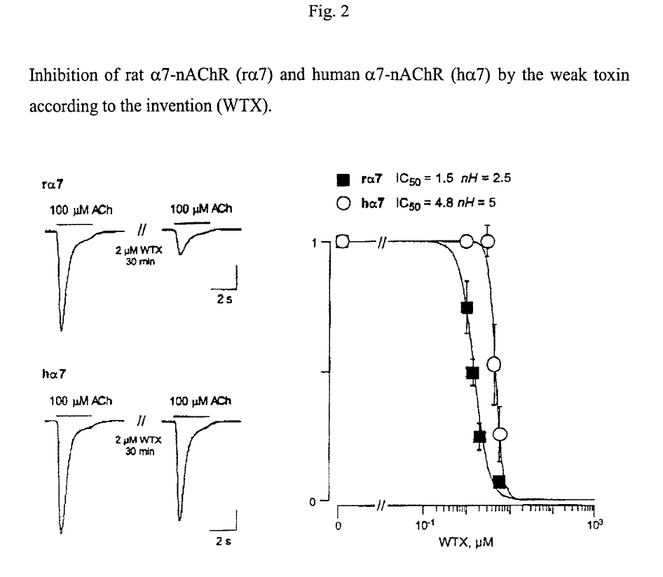

FIG. 2: The inhibition of rat α7-nAChR and of human α7-nAChR by the weak toxin according to the invention. The diagrams on the left depict typical currents produced by short ACh pulses (100 μM, 3s) by oocytes which express either rat α7 receptors (upper traces) or human α7 receptors (lower traces), before and after 30-minute treatment with 2 μM of the weak toxin according to the invention. The diagrams on the right represent dose-effect inhibition plots with the weak toxin according to the invention obtained by plotting the signal currents as a function of the toxin concentration. Values measured in different (2 to 4) cells were normalized to the control current induced by 100 μM ACh. The cells were kept at −80 mV.

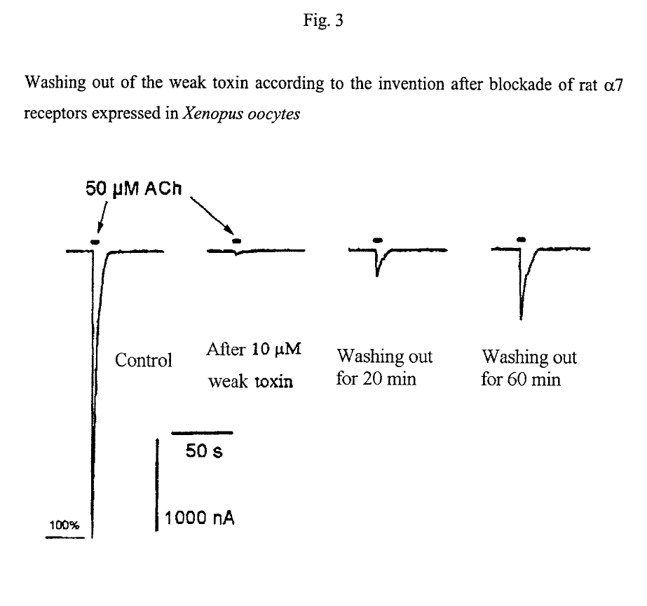

FIG. 3: The virtually irreversible blockade, shown by a washout experiment, of rat α7 receptors expressed in Xenopus oocytes. The currents measured with a 4 s application of 50 μM ACh at the start, after incubation in 10 μM of the weak toxin according to the invention for 20 min, and after washing out for 20 min and 60 min indicate a virtually irreversible blockade of the receptor by the weak toxin according to the invention.

The invention is illustrated further by preferred exemplary embodiments hereinafter but is not restricted thereto. Unless otherwise indicated, all amounts stated hereinafter refer to percentages by weight.

### EXAMPLES

1. Isolation of the weak toxin

Snakes of the species Naja kaouthia were kept in captivity and milked by manual massage of the venom gland. The venom was dried over anhydrous CaCl₂ and stored at −20° C.

200-300 mg of Naja kaouthia venom were fractionated on a Sephadex G-50sf (2.5×95 cm) column. The toxic main fraction was separated further on an HEMA BIO 1000 CM column (8×250 mm, from Tessek, Czechia) in an ammonium acetate gradient (pH 7.5) from 20 mM to 1 M. The fraction containing the weak toxin was finally purified by reverse phase HPLC on a Vydac C18 column (4.6×250 mm) in an acetonitrile/water gradient (from 15 to 45%) in the presence of 0.1% trifluoroacetic acid. The yield of weak toxin was about 1 mg.

The molecular mass of the toxin was determined by MALDI TOF using a BRUKER REFLEX (BRUKER) mass spectrometer. The toxin has a molecular weight of 7613 dalton.

The structure of the toxin was further determined by Edman degradation of proteolytic fragments using a 473A protein sequencer (Applied Biosystems, Foster City, Calif., U.S.A.). This unambiguously revealed the indicated the sequence of this toxin.

2. Detection of the interaction of the weak toxin with α7-nAChR by displacement of α-bungarotoxin binding

A solution of GST-α7-(1-209) fusion protein (17 μg/ml, pH 8.0, 0.1% CHAPS; cf. Ariel, S., Asher, O., Barchan, D., Ovadia, M., Fuchs, S., Ann. N.Y. Acad. Sci. 841 (1998), 93-96) which contains the ligand-binding domain of the alpha-7 nicotine receptor was incubated with various concentrations of the toxin to be tested in a liquid volume of 190 μl at room temperature for 1 h. Then 10 μl of 0.4 μM [¹²⁵I]α-bungarotoxin (aBgt) (prepared as described by Klukas, O., Peshenko, I. A., Rodionov, I. L., Telyakova, O. V., Utkin, Yu.N., Tsetlin, V. I., Bioorgan. Khim. 21 (1995), 152-155) were added, and the mixture was incubated for a further hour. The unbound [¹²⁵I]αBgt was removed by rapid filtration through DE-81 filters (Whatman, England) and the filters were washed four times with 1 ml of 50 mM Tris-HCl buffer (pH 8.0) with 0.1% Triton X-100 and counted using a γ counter (Ultragamma (LKB)). The weak toxin displaces α-Bgt in this experiment with an IC₅₀ of 4.3 μM, whereas an IC₅₀ of 9.1 μM is obtained with a-cobratoxin under these conditions (see FIG. 1).

3. Detection of the blockade of α7-nAChR bv the weak toxin with electrophysiological experiments in Xenopus oocytes

It is known and state of the art to detect the effect of agonists or antagonists of a nicotine receptor with electrophysiological methods. The corresponding methods and experimental designs are described in many places in the literature (see, for example, Kettenmann & Grantyn, eds. 1992). It is particularly simple and expedient in this connection for cloned receptor genes to be injected into xenopus oocytes and thus be expressed. The necessary electrophysiological measurements can then be carried out particularly simply and conveniently on these cells. (For example Bertrand D., Bertrand S., Ballivet M., Pharmacological properties of the homomeric alpha-7 receptor, Neurosci. Lett. 146, 87-90 (1992), Amar M., Thomas P., Johnson C., Lunt G. G., Wonnacott S., Agonist Pharmacology of the neuronal α7 nicotinic receptor expressed in Xenopus Oocytes, FEBS Lett. 327, 284-288 (1993), Cooper J. C., Gutbrod O., Witzemann V., Methfessel C., Pharmacology of the nicotinic acetylcholine receptor from fetal rat muscle expressed in Xenopus oocytes, Eur. J. Pharmacol. 309, 287-298 (1996)).

Xenopus oocytes were isolated and prepared as already described (Bertrand, D. et al., Methods in Neuroscience, 4 (1991), New York, Academic Press, 174-193). On the first day after isolation of the oocytes, 10 nl portions of a solution with 2 ng of an appropriate cDNA expression vector were injected into the cell nuclei of the oocytes. The oocytes were kept in a suitable medium (BARTH solution consisting (in mM) of NaCl 88, KCl 1, NaHCO₃ 2.4, MgSO₄ 0.82, Ca(NO₃)₂ 0.33, CaCl₂ 0.41, HEPES 10, pH 7.4) for 3-5 days. In order to minimize contamination, before use the medium was filtered at 0.2 μm and antibiotics were added (20 μg/ml kanamycin, 100 units/ml penicillin and 100 μg/ml streptomycin).

### Electrophysiological Experiments

Electrophysiological recordings were made using a dual electrode voltage clamp (GENECLAMP 500 from Axon Instruments, Forster Calif.) as already described [Bertrand D., Bertrand S., Ballivet M., Pharmacological properties of the homomeric alpha-7 receptor, Neurosci. Lett. 146, 87-90 (1992)]. Superfusion with OR2 oocyte ringer solution containing: 82.5 mM NaCl; 2.5 mM KCl; 2.5 mM CaCl₂; 1 mM MgCl₂; 5 mM HEPES; at pH 74 (adjusted with NaOH) was used for these electrophysiological experiments. Acetylcholine (Fluka, Buchs, Switzerland) was stored as stock solution at −20° C. and added to the OR2 immediately before the experiment. Weak toxin incubations were carried out by adding the toxin to the perfusion medium. In order to prevent adsorption of the toxin onto plastic surfaces, 20 μg/ml bovine serum albumin (Sigma, fraction V) were added to the solution.

One example of the blockade of α7-nAChR by the weak toxin is shown in FIG. 2. It is evident from the dose-effect plots that the toxin has an even stronger effect on rat α7-nAChR than on human α7-nAChR. FIG. 3 shows an example indicating that blockade of the α7-nAChR by the weak toxin is almost irreversible. Even after washing out the toxin for 60 minutes, the current induced by ACh remains far below the initial level.

4. Detection of the inhibition of the proliferation of SCLC cells

The proliferation of the cells can be quantified by culturing the cells in the culture medium by usual methods. After a suitable incubation time, the cells are to be dissociated, stained with a suitable dye and counted directly in a chamber (Schuller, H. Cell type specific, receptor mediated modulation of growth kinetics in human lung cancer cell lines by nicotine and tobacco-related nitrosamines, Biochemical Pharmacology 38, 3439-3442 (1989). It is possible in this way to demonstrate that nicotine or other agonists of α7-nAChR increase the rate of growth of these cells, whereas snake toxins such as alpha-cobratoxin, alpha-bungarotoxin or, in particular, weak toxin are able to slow down or suppress proliferation of these cells.

1. Peptide having the amino acid sequence
```
Leu Thr Cys Leu Asn Cys Pro Glu Met Phe Cys Gly Lys Phe Gln Ile
    1  5  10  15
Cys Arg Asn Gly Glu Lys Ile Cys Phe Lys Lys Leu His Gln Arg Arg
                          20  25  30
Pro Leu Ser Trp Arg Tyr Ile Arg Gly Cys Ala Asp Thr Cys Pro Val
                  35  40  45
Gly Lys Pro Tyr Glu Met Ile Glu Cys Cys Set Thr Asp Lys Cys Asn
          50  55  60
Arg  (I)
  65
```
or alleles derived therefrom, or peptides containing essential parts of the peptide (I).

2. Peptide according to claim 1, characterized in that up to five individual amino acids have been omitted, exchanged for other amino acids or replaced by short sequences of up to five other amino acids of any type.

3. Process for preparing a peptide according to claim 1 or 2, comprising isolation of the venom from snakes of the species Naja kaouthia by manual massage of the venom gland, drying of the venom over anhydrous CaCl₂, and isolation of the peptide by sequential ion exchange and reverse phase chromatography.

4. Peptide obtainable by the process according to claim 3.

5. Peptide according to claim 4, characterized in that it has a molecular weight of 7613 dalton.

6. Peptide according to claim 4 or 5, characterized in that it has the amino acid tryptophan in its primary sequence.

7. Pharmaceutical composition containing at least one peptide according to claim 1 or 4.

8. Use of a peptide according to claim 1 or 4 for producing a pharmaceutical for the treatment of diseases of which therapy is possible through inhibition of the α7-nACh receptor.

9. Use according to claim 8, characterized in that the disease is cancer.

10. Use according to claim 9, characterized in that it is small cell lung carcinoma (SCLC).

11. Use of a peptide according to claim 1 or 4 for producing a means for diagnosing cancer.

12. Use according to claim 11, characterized in that small cell lung carcinoma (SCLC) is diagnosed.

13. Method for labelling tumour cells in vitro, comprising the binding of a peptide according to claim 1 or 4 to the target cells.

14. Method according to claim 13, characterized in that the target cells are cells of small cell lung carcinoma (SCLC).
